(19) United States
(12) Patent Application Publication
(10) Pub. No.: US 2022/0175319 A1
(43) Pub. Date: Jun. 9, 2022

(54) IN VIVO IMMUNOASSAY SYSTEM

(71) Applicant: Given Imaging LTD, Yoqneam (IL)

(72) Inventors: Osnat Sella-Tavor, Kfar Kish (IL); Baruch Gruman, Lapid (IL)

(21) Appl. No.: 17/600,186

(22) PCT Filed: Mar. 31, 2020
(86) PCT No.: PCT/IL2020/050390

§ 371 Date: Sep. 30, 2021

Related U.S. Application Data
(60) Provisional application No. 62/827,463, filed on Apr. 1, 2019.

(51) Int. Cl. A61B 5/00; G01N 33/543; A61B 5/145; A61B 5/1455

(52) U.S. Cl. CPC A61B 5/6861; G01N 33/54388; A61B 5/14507; A61B 2562/0295; A61B 5/4238; A61B 5/4255; G01N 2800/06; A61B 5/1455

(57) ABSTRACT

A swallowable in-vivo device comprising a shell defining a cavity of the in-vivo device, the shell being formed with at least one aperture extending through the shell's wall. The in-vivo device is configured for allowing inlet of fluid into the cavity; The in in-vivo device further comprises and immunoassay system accommodated within the cavity and configured for interacting within the fluid; The in-vivo device also comprises at least one breach mechanism covering the at least one inlet for preventing ingress of fluids into the cavity via the inlet; The at least one breach mechanism comprises a film layer configured for reacting with the fluid and designed to be breached after a predetermined amount of exposure time to the GI fluid, corresponding to a desired location along the GI tract.

### TECHNOLOGICAL FIELD

The present invention relates to in vivo immunoassay in general, and to immunoassay using swallowable capsules in particular.

### BACKGROUND OF THE INVENTION

The basic principle of any immunochemical technique is that a specific antibody is combined with a specific antigen to give an exclusive antibody-antigen complex. Antigens are generally of high molecular weight and commonly are proteins or polysaccharides. Polypeptides, lipids, nucleic acids and many other materials can also function as antigens. Immune responses may also be generated against smaller substances, called haptens, if these are chemically coupled to a carrier protein or other synthetic matrices. A variety of molecules such as drugs, simple sugars, amino acids, small peptides, phospholipids, or triglycerides may function as haptens. Thus, assuming time is of no issue, about any foreign substance can be identified by the immune system and evoke specific antibody production.

Immunoassays are rapid, sensitive, and selective, and are generally cost effective. They have been applied to clinical diagnostics, environmental analysis and food safety assessment. Many types of immunoassay have been used to detect the presence of various substances, often generally called ligands, in body fluids such as blood and urine. Such assays involve antigen-antibody reactions, synthetic conjugates comprising radioactive, enzymatic, fluorescent, or visually observable metal sol tags, and specially designed reactor chambers. In these assays, there is a receptor, e.g., an antibody, which is specific for the selected ligand or antigen, and a means for detecting the presence, and often the amount, of the ligand-receptor reaction product. Most current tests are designed to make a quantitative determination, but in many cases all that is required is a positive/negative indication. For these tests, visually observable indicia such as the presence of agglutination or a color change are preferred.

Lateral flow immunoassay, which is also known as the immuno-chromatographic assay, or “strip” test, is an example of a widespread test that is simple to perform by almost anyone and operates more rapidly than traditional laboratory-based testing. This area of diagnostics has grown dramatically in recent years, with the most common and well-known of these being the home pregnancy test.

The principle of a lateral flow immunoassay relies on the competition for binding sites on polymer or metal particles. Antibodies that are raised to a specific target are bound to metal nanoparticles or dyed polymer particles. These particles are then applied using an immersion procedure onto a release pad (a sample pad) in order to produce a stable particle reservoir for release onto a nitrocellulose-based membrane. Two lines of reagents are immobilized onto, or formed or built into, the nitrocellulose-based membrane: a target reference, or test line, comprising a conjugate that can specifically bind the target to be identified, and (followed by) a spaced apart control line that is a line of anti-species antibody. The sample pad and membrane are assembled together with an absorbent pad. The sample is initially added to the adsorbent pad and the strip is left for a few minutes after which the result is visually read directly, looking for the coloration of the lines. This technology is ideally suited for rapid diagnostics.

Most medical detection kits utilizing the lateral flow immunoassay are based on in-vitro testing of body fluid, such as urine or blood. For example, in some cases, diseases, such as cancer, are detected by analyzing the blood stream for tumor specific markers, typically specific antibodies.

Formation of the detectable complexes at the test and control lines depend on the time period that the molecular components that should interact are sufficiently close in order to bind. The capillary flow rate determines the length of this interacting time period. Capillary flow rate decays exponentially as the liquids progress along the membrane. Reduced flow rate results in increased interaction time periods and increased effective/detectable analyte concentrations in the sample. Therefore, the location of the test line along the strip has a significant impact on achievable sensitivity. Due to this strip property, the common practice at lateral flow strips used for ex-vivo measurements in body fluids, is to locate the test band at the last 5 mm zone of the nitrocellulose membrane for highest performance. Lateral flow strips for ex-vivo measurements in body fluids samples (such as blood, stool, urine), are often designed to detect low biomarkers concentration, and if high concentrations should be detected, fluids can be diluted prior to the test.

Another example is the presence of elevated concentrations of red blood cells in the gastrointestinal (GI) tract that may indicate different pathologies, depending on the location of the bleeding along the GI tract. Thus, for instance, bleeding in the stomach may indicate an ulcer, whereas bleeding in the small intestine may indicate the presence of a tumor. Furthermore, different organs may contain different body fluids requiring different analysis methods. For example, the stomach secretes acids, whereas pancreatic juice is basic.

Thus, early in-vivo detection, identification and location of abnormal conditions (such as, for example, an atypical presence or concentration of a substance in body fluids) may be critical for definitive diagnosis and/or treating of various pathologies.

It is, therefore, an object of the present invention to provide a swallowable in-vivo device with on-board chromatographic strip that can provide rapid and sensitive in-vivo detection of low levels of various ligands, antigens or antibodies in body fluids. Another object of the present invention is to provide a swallowable in-vivo device with a chromatographic strip that is adapted to detect low levels of various ligands, antigens or antibodies in body fluids at various sites/locations in the GI tract.

There are different types of the lateral flow immunoassay available on the market. For example, in the double antibody sandwich immunoassay, the drawn body fluid migrates from a sample pad through a conjugate pad where any target analyte present will bind to the labelled conjugate particles. The sample fluid mixture then continues to migrate across the membrane until it reaches a test line, where the target/conjugate complex binds to the immobilized antibodies, producing a visible line on a membrane. The fluid then migrates further along the strip until it reaches the control line, where excess conjugate binds and produces a second visible line on the membrane. Control line is therefore indicative of the sample that has migrated across the membrane as intended. Thus, the two colored lines appearing on the membrane are a positive result. A single colored control line is a negative result. Double antibody sandwich assays are most suitable for larger analytes, such as bacterial pathogens and viruses, with multiple antigenic sites.

Competitive assays are primarily used for testing small molecules and differ from the double antibody sandwich immunoassay in that the conjugate pad contains labeling particles conjugated to the target analyte or to an analogue thereof. If the target analyte is present in the sample, it will not bind with the conjugate and will remain unlabelled. As the sample migrates along a reaction membrane and reaches the test line, an excess of unlabeled analyte will bind to the immobilized antibodies and block the capture of the conjugate, so that no visible line is produced. The unbound conjugate will then bind to the antibodies in the control line, producing a colored line. The single colored control line on the reaction membrane is a positive result. Two colored lines are a negative result. Competitive assays are most suitable for testing for small molecules, such as mycotoxins, unable to bind to more than one antibody simultaneously.

Lateral flow immunoassays are simple to use by untrained operators and generally produce a result within several minutes. The lines can take as little as a few minutes to develop. Generally, there is a tradeoff between time and sensitivity, such that more sensitive tests may take longer to develop. The lateral flow immunoassays typically require little or no sample or reagent preparation. They are very stable and robust, have a long shelf life and do not usually require refrigeration. They are also relatively inexpensive to produce. These features make them ideal for use in the in vivo diagnostic device according to the embodiments of the invention.

There are also known in-vivo swallowable devices comprising a lateral flow immunoassay systems, configured for detecting various components in the GI tract.

Acknowledgement of the above references herein is not to be inferred as meaning that these are in any way relevant to the patentability of the presently disclosed subject matter.

### SUMMARY

In accordance with a general aspect of the subject matter of the present application, there is provided a miniaturized LFS (lateral flow strip) modified for being contained in a swallowable capsule configured for traveling along the GI tract and being able to draw-in body fluids in a controlled manner and provide useful biological-related measurements while in the gastrointestinal (GI) tract.

In accordance with one aspect of the subject matter of the present application there is provided a swallowable in-vivo device comprising:
a shell defining a cavity of the in-vivo device, said shell being formed with at least one aperture extending through said shell's wall, and configured for allowing inlet of fluid into said cavity;
an immunoassay system accommodated within said cavity and configured for interacting within said fluid; and
at least one breach mechanism covering said at least one inlet for preventing ingress of fluids into said cavity via said inlet, said at least one breach mechanism comprising a film layer configured for reacting with said fluid and designed to be breached after a predetermined amount of exposure time to said GI fluid, corresponding to a desired location along the GI tract.

The term ‘breach’ used herein should be understood as defining a condition in which at least a portion of the breach mechanism no longer covers the at least one inlet and allows ingress of fluid into the cavity.

In accordance with one example, the breach mechanism may be constituted by the film layer, covering said at least one inlet, and configured for becoming dissolved after a predetermined amount of time. In accordance with another example, the film layer constitutes part of a mechanism configured for attaching a cover of the breach mechanism to the shell for covering the inlet, wherein, when the film layer is eroded, the cover detaches from the shell, thereby exposing the opening.

The film layer may be formed as a stand-alone component before being assembled/fitted to the shell of the in-vivo device. Specifically, the film layer may be manufactured separately from the in-vivo device and thereafter be adhered to the shell during assembly.

In accordance with the present invention, the film layer is predesigned to become sufficiently eroded over a given period of time. Such a design may be facilitated by a plurality of design parameters such as, but not limited to, the shape, dimensions and composition of the film (e.g. the amount of reactive material).

It should be noted that the film layer is designed to function as a cut-off barrier, i.e. preventing ingress of fluid into the inlet before it is breached. Specifically, the film layer is configured for preventing ingress of fluid into the inlet by any way other than breach of the film layer. This is contrary to mechanisms which may allow slow seepage or diffusion of fluid (low flow rate) into the inlet and thereafter become breached.

In accordance with a specific example, the film layer may comprise a combination of at least the following materials:
a cut-off active material having a threshold response to a specific substance of the GI fluid or to a specific parameter of the GI fluid;
a plasticizer configured, together with the cut-off sensitive material, for forming said film layer; and
an auxiliary active material.

The cut-off active material may be an enteric material configured for reacting with the GI fluid under/over a given pH level. Such a material will remain inactive as long as the pH level is under/over a given threshold, such that the film layer does now continuously and gradually allow intake/diffusion of GI fluids through the film, but rather becomes breached given the proper pH level.

In accordance with a specific example, the film layer may contain between 40-98% of enteric material, even more specifically, between 45-97% enteric material, and even more specifically, between 50-95% of enteric material.

The plasticizer may be from the triethyl citrate family of materials or, alternatively, a glycol, diesters and triesters of acids (such as Triethyl citrate, Tributyl citrate, Acetyl triethyl citrate, Acetyl Tributyl Citrate, Dibutyl sebacate, Diethyl Phthalate, di butyl phtalate), diesters and triesters of alcohols (such as Triacetin, Tributyl Citrate, Triethyl Citrat), natural oils (such as Vegetable oils, Fractionated coconut oil, Acetylated monoglycerides), Polyethylene Glycols, Polyethylene Glycol Monomethyl Ether, Castor Oil, Propylene Glycol, Diacetylated Monoglycerides, Sorbitol, Sorbitan Solution, Glycerin. In accordance with a particular example, the plasticizer may be propylene glycol or polyethylene glycol (PEG). The amount of plasticizer in the film layer may be the complement of the enteric material to 100%, i.e. for an X % of enteric material, the amount of plasticizer will be Y %=100−X. Thus, the amount of plasticizer in the film layer may range from 60-2%, more specifically, 55-3% and even more particularly, 50-4%.

The auxiliary active material may be configured for providing the film layer with additional resilience, making it less brittle or prone to crack/break during handling and in the GI fluid environment, in order to prevent breach of the film layer in inappropriate GI conditions. In addition, the auxiliary active material may be a hydro-swelling enteric polymer configured for retaining fluid within the film layer before its breach. Specifically, as the auxiliary active material reacts with the GI fluids, it may degrade the structure of the film layer, retaining fluid therein without breaching the film. This yields that, when the active cut-off material of the film eventually reacts with the GI fluid, the breach of the film layer happens very quickly, almost instantly, as the entire film's composition has, by that time, a high amount of fluid.

It should be noted that using the film layer having the above described breach mechanism may be useful not only for allowing intake of fluids into a swallowable device for the purpose of immunoassay, but even as a simple indicator that the in-vivo device has reached a given section of the GI. Specifically, the in-vivo device may comprise a sensor behind said film layer, and the film layer may be designed to become breached at a certain location along the GI tract, wherein, when the film layer is breached, the fluid can trigger the sensor, thereby indicating that the in-vivo device has reached a desired location.

In accordance with a specific example, the amount of auxiliary active material may be provided as a given percentage of the overall weight of the film layer, and, specifically, as a given percentage of the combined weight of the active cut-off material and the plasticizer. Under the above arrangement, the amount of auxiliary active material may range between 2-40%, more specifically, between 3-35%, and even more specifically, between 5-30% of said combined weight.

Within the above given ranges, the difference between different ratios and combinations of the cut-off active material, plasticizer and auxiliary active material may allow designing the film to become breached under different GI conditions, thereby allowing to tailor the film layer to become breached in a specific location of the GI tract, based on the knowledge of the conditions of the GI fluids in said specific location.

The film layer may have a covering area juxtaposed with the inlet, e.g. the projection of the shape of the inlet on the film layer when the film layer is overlaid on the inlet, and a peripheral area juxtaposed with a portion of the in-vivo device, e.g. the shell.

In particular, the thickness of the covering area of the film layer (measured perpendicular to a plane of the inlet) may also be used as a means for controlling the amount of time required for breaching the breach mechanism—the greater the thickness, the longer it would take for the film layer to become breached. Since the above mentioned parameters of the GI fluid are in predetermined ranges within the body, the thickness of reactive material may also be calibrated to tailor the film layer to allow breach of the film layer in a specific section of the GI tract.

For example, in the case of pH, the film layer may be designed to dissolve in the presence of a pre-defined pH level within a time frame that is based on the capsule device transit time in the GI.

This performance is achieved by using pH dissolving polymers in combination with other polymers known to erode/dissolve upon exposure to aquatic media, as well as by control over the thickness of the films. Potentially, enzymatic and/or microfloral targets (such as amylose, pectin, polysaccharides and other natural occurring polymers) may be incorporated into the film in order to prevent premature film dissolution on the one hand and allow for dissolution in lower pH on the other.

pH dissolving polymers may be any of the following types: polyanions polymers (dissolving in increased PH) or polycations (dissolving in lower PH). These group of polymers includes (but are not limited to) poly acrylate and derivatives, poly methacrylate and derivatives, Cellulosic polymers and derivatives, polyacrylamide and derivatives, poly(ethylene imine) and derivatives, poly(L-lysine) and derivatives, chitosane and modifications of it, polyethylene glycol and modifications of it, polypropylene glycol and modifications of it, polyethylene oxide and modifications of it, polyurethanes and modifications of it, albumin and modifications of it, polyesters and modifications of it, hydroxyproline, poly(vinylpyridine) and derivatives, poly(vinylamine) and derivatives, gelatin and derivatives, polyvinylacetates and modifications of it, starch and derivatives, pectin, alginates.

These polymers may be used as homopolymers and/or as copolymers of various monomers and in all variations of structure (block copolymers, periodic copolymers, alternating copolymers, grafted copolymers or random copolymers).

These polymers and derivatives can be mixed in the formulation with any other polymer/s and excipients, to allow for film formation (including plasticizers, lubricants, film forming reagents, salts, disintegrants, solubilizing reagents, functionally added excipients).

In accordance with another aspect of the subject matter of the present application there is provided a film layer configured for being used in an in-vivo device of the previous aspect of the present application, said film layer containing a cut-off active material having a threshold reaction to a specific substance or parameter of the GI fluid, a plasticizer configured, together with the cut-off sensitive material, for forming said film layer, and an auxiliary active material, and wherein the film layer is designed to be breached after a predetermined amount of time of exposure to said fluid.

In accordance with yet another aspect of the subject matter of the present application, there is provided a lateral flow strip comprising a fluid intake end and a distal end, and at least one test band located closer to said fluid intake end than to said distal end.

The lateral flow strip may be divided into an intake section including the fluid intake end, and intermediate section and a distal section comprising the distal end. The arrangement may be such that the at least one test band is located in the first section or in the intermediate section.

The lateral flow strip of the in-vivo device of the present application is configured for coming in direct contact with GI fluid, in-vivo, which contain a high concentration of biomarkers owing to their proximity to GI lesions. Providing a lateral flow strip comprising a test band located proximal to the fluid intake end allows obtaining an accurate measure of the biomarkers, without diluting the GI fluid.

Thus, the lateral flow strip of the in-vivo device of the present invention allows a valid quantitative detection of biomarkers despite reagent saturation. In addition, since the lateral flow strip is configured for being used in a swallowable and ingestible in-vivo device which has limited dimensions, providing the test band in the first third of the lateral flow strip allows significantly reducing the overall length of the strip, as portions of the intermediate segment and distal segment of the lateral flow strip may be reduced in length without affecting the at least one test band.

In particular, in lateral strips ranging between 20-40 mm in length, the at least one test band in accordance with the present invention may be located at the first 5-13 mm of the lateral flow strip. In such a location close to the strip, origin flow rate is higher, time period for complexes formation is shorter, and thus effective Ag concentration decreases—thereby eliminating the need for sample dilution to reduce the effective detectable Ag concentration. Such strip structure also enables the production of a very short lateral flow strip that can be accommodated within ingestible capsule device.

In accordance with still another aspect of the subject matter of the present application, there is provided a lateral flow strip extending between a fluid intake end and a distal end, said strip comprising a sample pad and a reagent pad, both proximal to the fluid intake end, a test pad comprising at least one test band, and an absorbent pad proximal to the distal end, wherein the ratio between the overall length of the lateral flow strip and the cumulative length of the reagent pad and said at least one test band is in the range of 3-6.

In accordance with another aspect of the present invention there is provided a swallowable in-vivo immunoassay device, said device comprising:
a lateral flow strip extending between a fluid intake end and a distal end, said strip comprising a test pad, and a backing card juxtaposed with said at least one test pad, wherein said backing card is made of a material allowing at least partial passage of light therethrough;
an illumination module comprising at least one illumination source configured for directing light towards said backing card; and
a sensor module comprising at least one sensor configured for receiving light from said illumination module, wherein said at least one sensor is positioned such that the test pad is disposed between the at least one sensor and said backing card.

The above arrangement allows illumination of the test pad through said backing card, with said at least one sensor receiving light from the light source after it had passed through the test pad. It is also appreciated that since, during the immunoassay process, the test pad is configured for changing its property (e.g. color) upon a physical/chemical reaction, passing light through the test pd may allow the sensor to sense the change in said property.

The test pad may comprise one or more test bands therealong, configured for changing at least one of their properties upon a chemical/physical reaction with the GI fluids. It is appreciated that when the test pad is formed with such one or more test bands, the bands are configured for reacting with the GI fluids while areas of the test pad free of the bands are configured either not to react with the GI fluid or react differently than the bands such that there's a clear difference in said property between the test bands and the test pad.

In accordance with one design embodiment, the direction of the light emitted from the light source may be generally transverse to the backing card, with the light piercing the backing card, impinging on the test pad and eventually being picked up by the at least one sensor.

In accordance with another design embodiment, the direction of the light emitted from the light source may be oriented generally along the backing card (e.g. from an end thereof), so that it travels along the backing card. Under this example, the backing card may comprise light directing elements configured for manipulating the direction of light for it to impinge on the test pad. Such light directing elements may be grooves, slits, scratches, imperfection or any other formation within the transparent backing card which will cause a change in the direction of the light beams emitted from the source. These light direction elements can be prefabricated within the transparent backing card or formed thereon after its manufacture.

Without such light directing elements, the majority of light is likely to travel along the backing card and simply be emitted through the other end thereof. However, it should be noted that even without these light directing elements, light may still change direction with the backing card and impinge on the test pad, albeit with poorer results than with light directing elements.

The light directing elements may be arranged along the length of the backing card, at least proximal to the areas juxtaposed having a test band thereon, in order to insure that light impinges on said test band/s for the purpose of identifying the result of the immunoassay process. In accordance with a specific example, the majority of the backing card may be provided with such light directing elements, while, in accordance with another example, the light directing elements are limited to area juxtaposed with the test bands.

In addition, the backing card may have a thickness t measured normally to the backing card. The distance of light directing elements from the test pad may vary based on their location along the backing card. Specifically, in accordance with a particular example, the light directing elements located proximal to the light source may be located the farthest from the test pad (e.g. maximal distance of t) while the light directing elements located distal from the light source may be located the nearest to the test pad. Depending on the arrangement of the test bands and/or on other requirements, the distance of the light directing elements from the test pad may vary continuously or discretely.

The sensor module may comprise one or more sensors, each sensor being configured for being juxtaposed with a certain test band of the test band, so as to receive light therefrom. The sensor module may also comprise at least one reference sensor juxtaposed with a portion of the test pad which is free of a test band, serving as a baseline light measurement.

It should also be pointed out that the above described arrangement may allow detecting ingress of fluid into the in-vivo device, even if the test pad is not formed with any bands. Specifically, the test pad may change its color, opacity etc. when becoming soaked with the GI fluids, a change which may be detected by the sensor. Thus, the above arrangement may also be used as a breach detector of the in-vivo device.

It was noticed that during the immunoassay process, the chemical reaction of the test bands with the GI fluids yields a distinct color, wherein measuring the Green to Red (G/R) allows clearly determining if a test band has chemically reacted as required. Specifically, such a G/R test will yield a baseline value when measured by the reference sensor, and an increased value when measured by a sensor juxtaposed with a test band. Thus, the above arrangement provides a simple and elegant method of reading the test bands.

Specifically, since the value in question is a simple ratio, it may elegantly eliminate the need of obtaining an image of the test pad and thereafter analyzing said image in order to determine if proper immunoassay reaction has taken place. In addition, removing the need for obtaining and analyzing an image may allow reducing the size of the sensors used in the in-vivo device, which may provide a significant advantage since in-vivo devices are, by definition, limited by space and dimensions.

Thus, in a accordance with another example of the subject matter of the present application, there is provided a system for obtaining an immunoassay reading from the lateral flow strip of an in-vivo device, said system comprising a light source configured for illuminating a test band of the lateral flow strip, at least one sensor configured for receiving light that has impinged on or passed through said test band, and at least one processor configured for calculating a value for the ratio R between two different wavelengths of the received light.

It should also be noted that as light travels through the transparent backing card, the absolute light intensity thereof decays significantly. Thus, using a unitless value such as a the ratio R as in the present invention, may allow normalizing values such that they are not affected by the light intensity level. Specifically, as in the previous example, the ratio between the red wavelength and the green wavelength remains essentially the same between different bands despite the fact that light passing through bands located closer to the light source will exhibit a higher absolute light intensity than light passing through bands located farther from the light source.

In accordance with still another aspect of the subject matter of the present application, there is provided a method for obtaining an immunoassay reading using the system of the previous aspect, said method comprising at least the steps of:
a) illuminating a test band of a lateral flow strip;
b) obtaining the light returned from or passed through said test band;
c) calculating a ratio R between two different wavelengths of the received light; and
d) comparing said value to a baseline value.

In accordance with yet another aspect of the subject matter of the present application there is provided a swallowable in-vivo device configured for performing an immunoassay and accommodating therein at least one lateral flow strip, said in-vivo device having an outer shell comprising a first end piece, a second end piece and an intermediate ring piece interposed between the end piece.

Each of the first and second end pieces may have a peripheral rim and the intermediate ring piece may have a first peripheral rim configured, in an assembled position of the in-vivo device, for being mated against the peripheral rim of the first end piece, and a second peripheral rim configured, in an assembled position of the in-vivo device, for being mated against the peripheral rim of the second end piece.

In accordance with a specific design embodiment, the in-vivo device may comprise two or more intermediate ring pieces, allowing a modular arrangement of the in-vivo device. The lateral flow strip may be accommodated within the intermediate ring piece/s of the in-vivo device, extending peripherally therealong, so that the lateral flow strip extends circumferentially around a longitudinal axis of the in-vivo device.

Thus, the modular arrangement of the intermediate ring pieces allows using a plurality of lateral flow strips, each being accommodated within its own intermediate ring piece, and arranging their order in accordance with design requirements. However, it should be appreciated that, under another example, an intermediate ring piece may also accommodate therein two or more lateral flow strips. Under any of the above arrangements, the number of lateral flow strips and/or the number of ring pieces only affects the length of the in-vivo device, but not its diameter.

At least each of the intermediate ring pieces accommodating therein a lateral flow strip may be formed with a gate configured for allowing ingress of fluid into the in-vivo device, in order to be absorbed by the lateral flow strip.

One advantage of the above suggested arrangement lies in the assembly process of the in-vivo device, allowing convenient access to each shell piece for fitting the lateral flow strip thereto. Specifically, in assembly, a shell piece may be fitted with a lateral flow strip and only thereafter, all the shell pieces may be assembly to form the shell of the in-vivo device.

In accordance with a further aspect of the subject matter of the present application, there is provided a swallowable in-vivo device configured for performing an immunoassay and accommodating therein at least one lateral flow strip, said in-vivo device having an outer shell comprised of three or more shell pieces, each shell piece extending along a longitudinal axis of the in-vivo device and having a first dome section and a second dome section, wherein the first dome sections of the shell pieces form together a first end dome of the in-vivo device and the second dome sections of the shell pieces form together a second end dome of the in-vivo device.

In accordance with a specific example, the at least one lateral flow strip is entirely accommodated within one of the shell pieces. Furthermore, the in-vivo device may comprise two or more lateral flow strips, in which case, each shell piece may fully accommodate one or more of the lateral flow strips therein.

One advantage such an arrangement may provide, inter alia, is accommodating the lateral flow strip/s within the in-vivo device with minimal bending thereof, as they extend along the length of the entire strip. Another advantage lies in the assembly process of the in-vivo device, allowing convenient access to each shell piece for fitting the lateral flow strip thereto. Specifically, in assembly, a shell piece may be fitted with a lateral flow strip and only thereafter, all the shell pieces may be assembly to form the shell of the in-vivo device.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic view of an immunoassay system in accordance with embodiments of the present invention;

FIG. 1B is a schematic view of another example of an immunoassay system in accordance with embodiments of the present invention;

FIG. 1C is a schematic view of still another example of an immunoassay system in accordance with embodiments of the present invention;

FIG. 1D is a schematic view of yet another example of an immunoassay system in accordance with embodiments of the present invention;

FIG. 1E is a schematic enlarged view of a detail A shown in FIG. 1D in accordance with embodiments of the present invention;

FIG. 2 is a schematic plot of values measured by the sensor of the immunoassay system of embodiments of the present invention;

FIG. 3A is a schematic isometric view of an in-vivo device according to embodiments of the present invention;

FIG. 3B is a schematic isometric cross-section view of the in-vivo device shown in FIG. 3A, taken along a plane perpendicular to its longitudinal axis in accordance with embodiments of the present invention;

FIG. 3C is a schematic isometric cross-section view of the in-vivo device shown in FIG. 3A, taken along its longitudinal axis in accordance with embodiments of the present invention;

FIG. 3D is a schematic isometric view of a single ring used in the construction of the in-vivo device shown in FIGS. 3A to 3C in accordance with embodiments of the present invention;

FIG. 4A is a schematic isometric view of a breach film in accordance with the embodiments of present invention;

FIG. 4B is a schematic cross-section view of the breach film shown in FIG. 4A, shown prior to its breach in accordance with embodiments of the present invention;

FIG. 4C is a schematic cross-section view of the breach film shown in FIG. 4B, shown in a breached condition in accordance with embodiments of the present invention;

FIG. 5C is a schematic isometric view of an in-vivo device in accordance with another example embodiment;

FIG. 5B is a schematic isometric view of the arrangement of strips within the in-vivo device of FIG. 5B in accordance with embodiments of the present invention;

FIG. 5C is a schematic isometric longitudinal cross-section view of the in-vivo device shown in FIG. 5A in accordance with embodiments of the present invention;

FIG. 6A is a schematic isometric view of an in-vivo device in accordance with a variation on the in-vivo device shown in FIGS. 5A to 5C in accordance with embodiments of the present invention;

FIG. 6B is a schematic longitudinal cross-section view of the in-vivo device shown in FIG. 6A in accordance with embodiments of the present invention;

FIG. 6C is a schematic enlarged view of a detail B shown in FIG. 6B in accordance with embodiments of the present invention;

FIG. 7A is a schematic isometric view of an in-vivo device in accordance with another example embodiment of the present application;

FIG. 7B is a schematic isometric view of the arrangement of lateral flow strips within the in-vivo device shown in FIG. 7A in accordance with embodiments of the present invention;

FIG. 7C is a schematic longitudinal cross-section view of the in-vivo device shown in FIG. 7A in accordance with embodiments of the present invention;

FIG. 8A is a schematic isometric view of an in-vivo device in accordance with another example embodiment of the present application; and

FIG. 8B is a schematic isometric view of a shell piece of the in-vivo device shown in FIG. 8A in accordance with embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS

In the following detailed description, numerous specific details are set forth in order to provide a thorough understanding of the invention. However, it will be understood by those skilled in the art that the present invention can be practiced without these specific details. In other instances, well-known methods, procedures, and components, modules, units and/or circuits have not been described in detail so as not to obscure the invention.

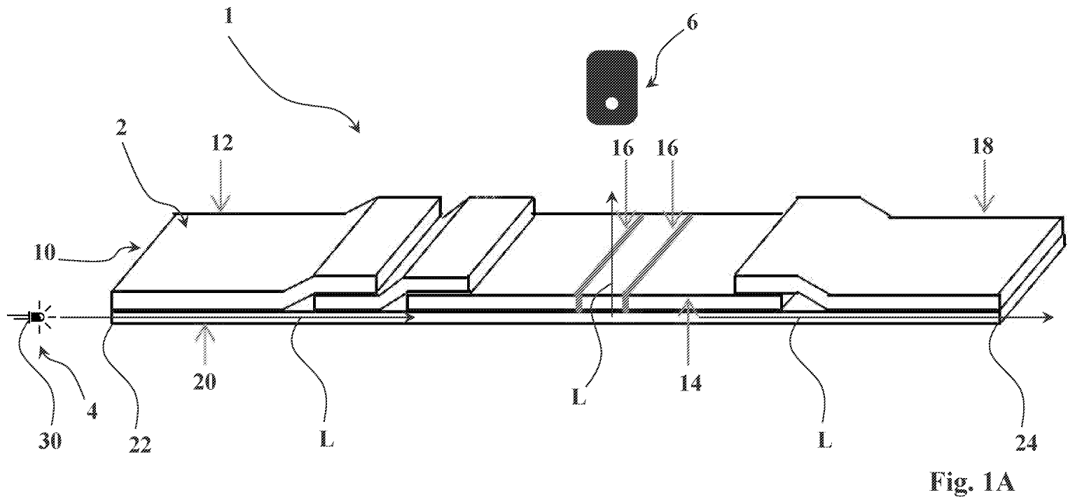

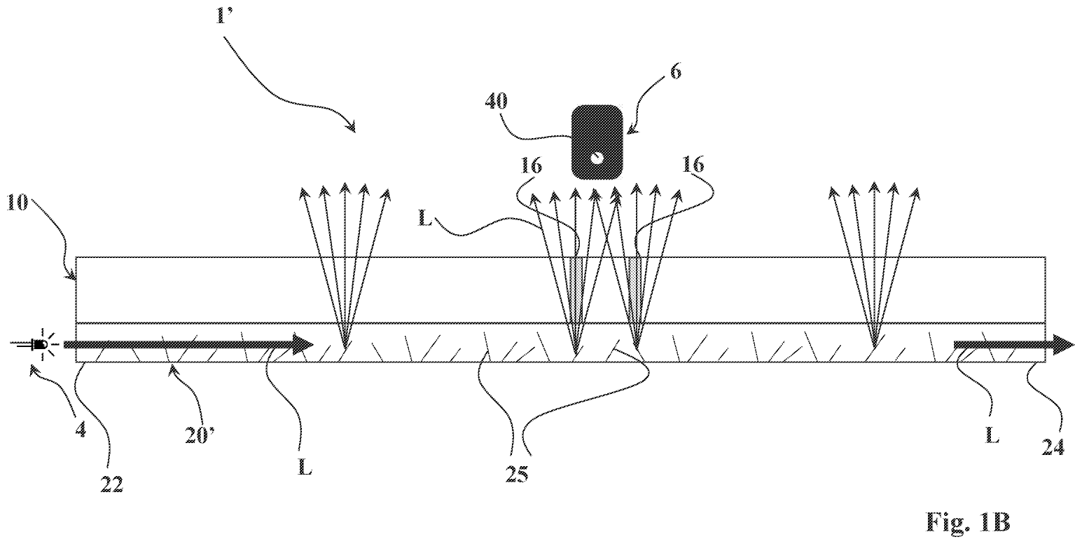

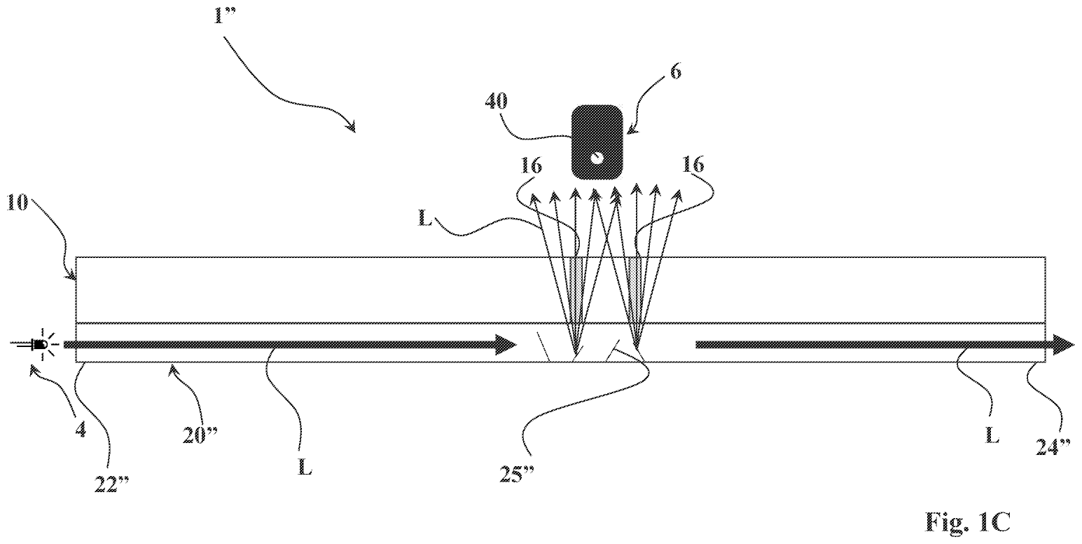

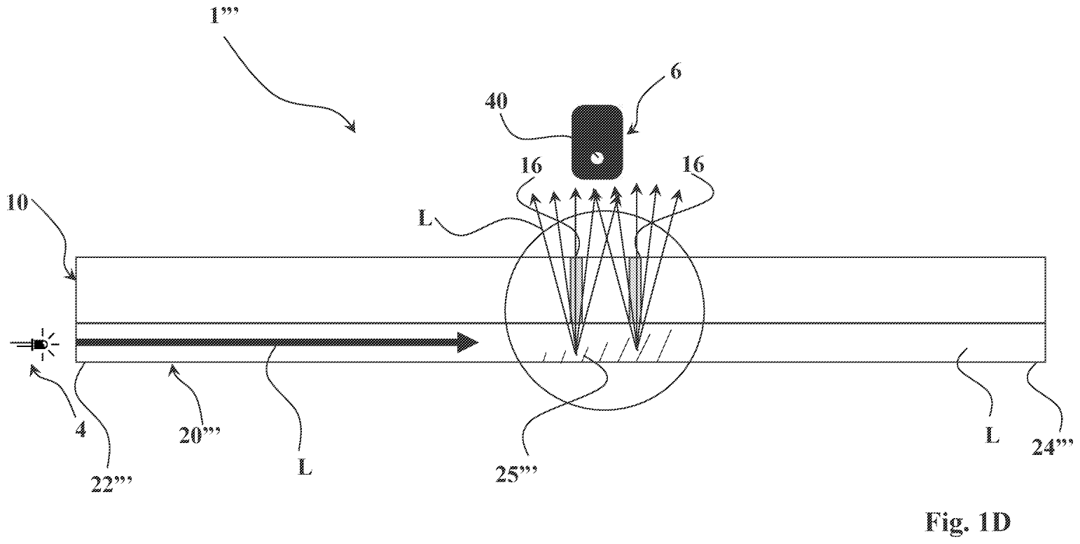

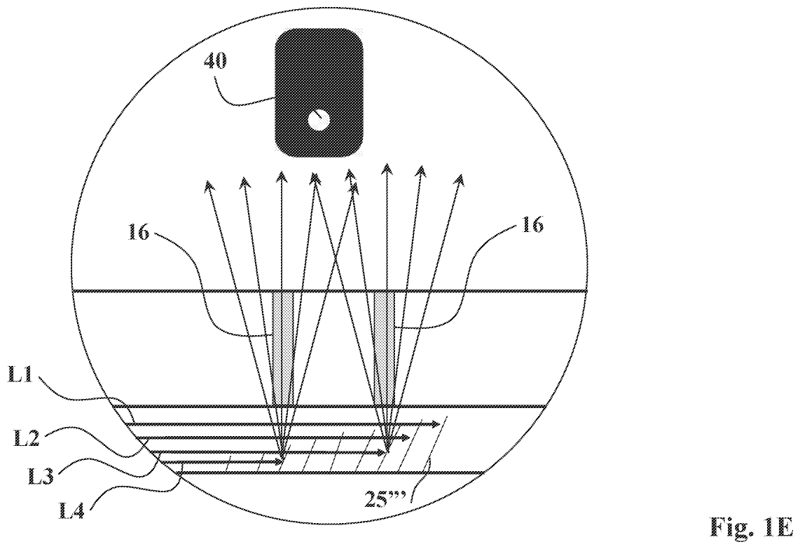

Attention is first drawn to FIG. 1A in which an immunoassay system is shown, generally designated 1 and comprising a lateral flow strip 2, a light source 4 and a sensor module 6. The lateral flow strip 2 comprises a functional portion 10 and a transparent backing card 20. The functional portion 10 comprises sample pad 12, a test zone 14 with several test bands 16, and an absorbent pad 18, as known per se. The backing card 20 has a first end 22 configured for receiving light therein, and a second end 24.

The light source 4 comprises an illumination element 30 configured for emitting light into the first end 22 of the transparent backing card 20. The sensor module 2 is disposed on the other side of the lateral flow strip 2, opposite the backing card 20, and comprises a sensor 40 configured for collecting light passing through the functional portion 10 of the lateral flow strip 2.

As shown in FIG. 1A, when light L is directed to the transparent backing card 20, it enters the first end 22 and passes freely through the backing card 20 resulting in the majority of the light being emitted through the second end 24, with only a small fraction of the light L being directed to the functional portion 10. A certain percentage of that small fraction of light L will be collected by the light sensor 40.

Turning now to FIG. 1B, another configuration of the immunoassay system is shown, generally designated 1′, in which the backing card 20′ comprises light directing elements 25 in the form of slits/grooves. In this configuration, light L entering the transparent backing card 20′ passes therethrough, and instead of progressing freely, encounters the light directing elements 25, causing dispersion of the light L in all directions. Therefore, in the current example, a considerably greater fraction of the light L is directed towards the functional portion 10, thereby also increasing the amount of light picked up by the sensor 40.

It is noted that in the present example, the light directing elements are formed along the entire backing card 20′ in order to cause maximal dispersion of the light L, to increase the amount of light L which can be picked up by the sensor 40.

Turning now to FIG. 1C, another example of the immunoassay system is shown, generally designated 1″, in which the backing card 20″ also comprises light directing elements 25″, with the difference being that these elements 25″ are formed adjacent the test bands 16 of the functional part 10. Thus, when light L is provided to the transparent backing card 20″, it will only undergo dispersion when encountering the light directing elements 25″ at the vicinity of the test bands 16. Thus, the areas of the test bands 16, which are the areas of interest for the sensor, will become more illuminated, while the regions between the test bands 16 will be less illuminated. This configuration may allow a clearer view of the test bands 16.

Attention is now drawn to FIGS. 1D and 1E, in which yet another example of the immunoassay system is shown, generally designated 1′″, in which the backing card 20′″ is formed with light directing elements 25′″ which are formed with increasing depth along the backing card 20′″. Specifically, in an area proximal to the light source 30, the light directing elements 25′″ extend into a shallow depth of the backing card 20′″, while in an area distal from the light source 30, the light directing elements 25′″ extend deeper into the backing card 20′″. The depth of the light directing elements 25′″ increases continuously.

One advantage of this configuration is that the light directing elements 25′″ proximal to the light source 30 do not disperse the entire amount of light L entering the backing card 20′″, but rather only the light L4 progressing via an area distal from the functional portion, thereby allowing light L1 progressing via an area proximal to the functional portion to progress and reach an area distal from the light source 30. Such an arrangement may provide a better illumination of the lateral flow strip.

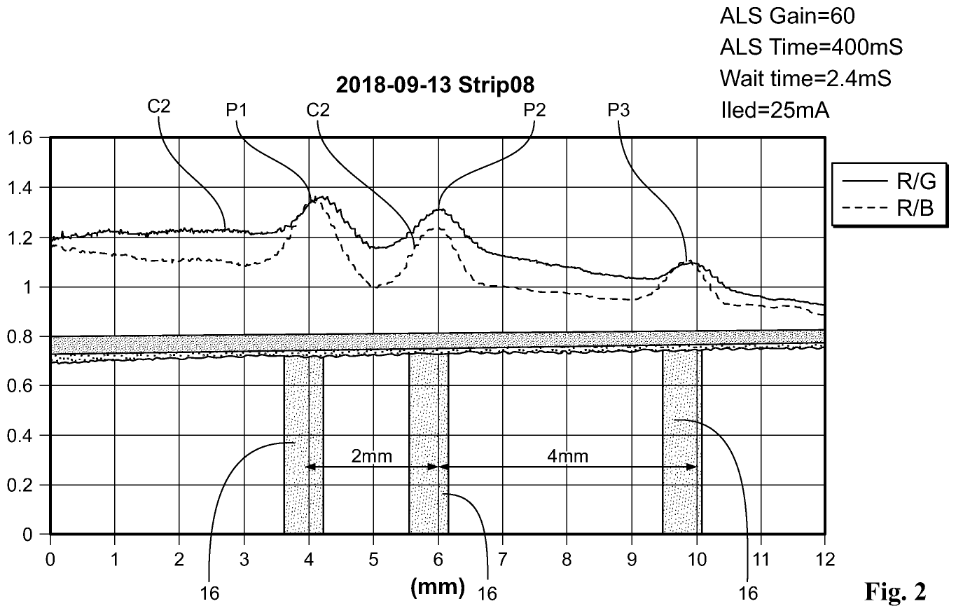

Turning now to FIG. 2, a chart is shown, plotting the ratio of Red to Green (R/G) wavelengths, denoted as C1 and the Red to Blue (R/B) wavelengths, denoted as C2, as registered by the sensor 40 from the strip LFS. The chart is overlayed on the strip itself for purpose of clarity. It is noted that while the absolute light intensity decays as light L progresses through the backing card 20, using a ratio allows normalizing the values such that they are not affected by said light intensity. It is clearly seen from the plotted chart where the bands of the test zone are located, corresponding to the peaks P1, P2 and P3 of each plot C1 and C2.

Furthermore, it is also demonstrated that in the specific example of the lateral flow strip LFS tested, the ratio of R/G provides a slightly more distinct indication of the location of the bands than the ratio R/B. However, is should be noted that each LFS may have a unique preferred ratio based on the color change undergone by the test bands located on the strip LFS.

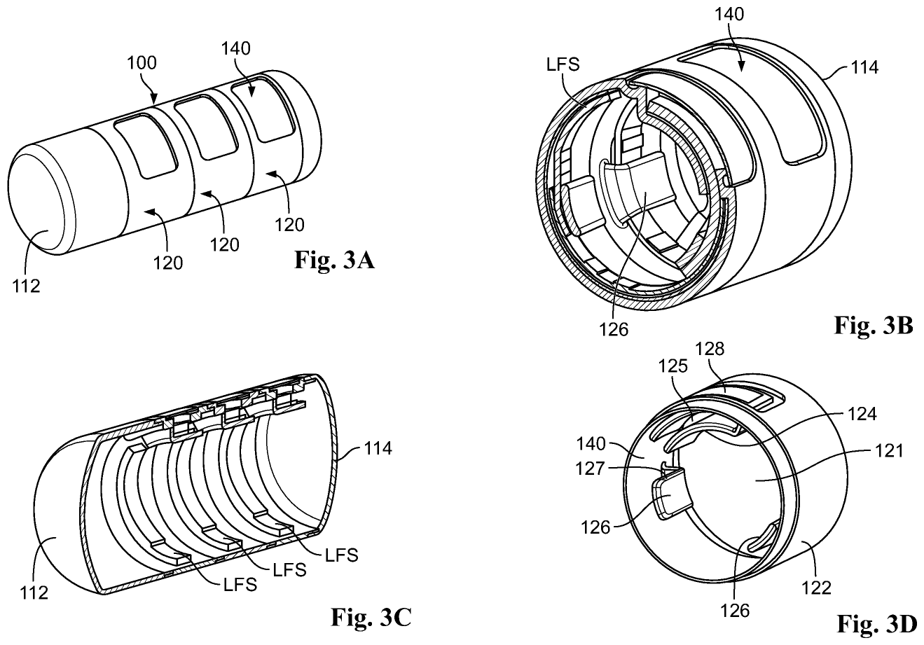

Attention is now drawn to FIGS. 3A to 3D, in which an in-vivo device is shown, generally designated as 100 and comprising a shell 101 assembled from a first end cap 112, a second end cap 112, and three ring pieces 120. The in-vivo device 100 further comprises three lateral flow strips LFS, each accommodated within one of the ring pieces 120, and three corresponding breach gates in the form of thin films 140, closing an opening allowing ingress of GI fluids into the shell 101.

Each ring piece 120 is formed as a cylindrical shell 122 defining therein a cavity 121. The inner wall of the shell 122 is formed with a primary holder 124 and two auxiliary holders 126 spaced from the inner wall and defining corresponding primary and auxiliary slots 125 and 127, into which the LFS can be fitted. Under the present example, each LFS is inserted into the slots 125 and 127 to extend circumferentially about the inner wall of the shell 122.

The width and diameter of the ring pieces 120 is based on the width and length of the LFSs, specifically, the ring piece is at least as wide as the LFS and its inner circumference is at least as long as the LFS. However, it should be understood that other designs may be possible in which each ring piece 120 holds more than on LFS, side by side (i.e. having a width equivalent to two LFSs or more).

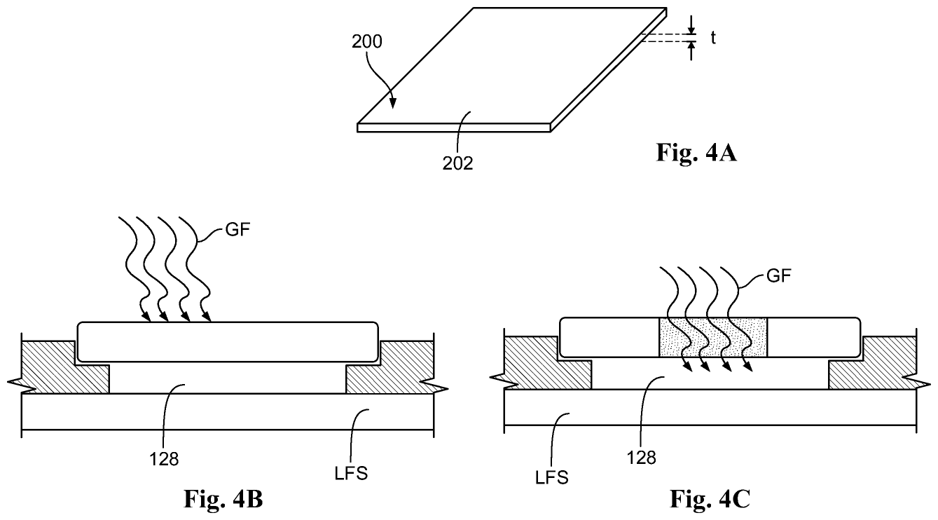

Each ring piece 120 is further formed with an inlet 128 configured for allowing ingress of GI fluids into the cavity of the ring piece 120 to come into contact with the LFS. Each such inlet 128 is sealed off with a thin film 140, preventing such ingress of fluids except under specific conditions as will be discussed with respect to FIGS. 4A to 4C.

Thus, each ring piece 120, when fitted with its corresponding LFS and sealing film 140, constituted a modular unit of the in-vivo device 100. In the current example, the in-vivo device comprises three such modular units, but it is appreciated that since they are modular, the in-vivo device 100 may comprise more or less ring pieces 120, according to specific requirements, the number of ring pieces and their width defining the length of the in-vivo device. It should also be noted that for in-vivo swallowable use, the length is limited by the side swallowable by a person.

The shell 101 in-vivo device 100 defines an inner cavity configured for accommodating therein the additionally required mechanical/electrical components of the in-vivo device (not shown), as known per se.

In operation, when the breach film is dissolved, GI fluid enters the inner cavity 121 of the ring piece 120, coming into contact with the LFS and allowing performing an immunoassay process by reacting with the materials of the LFS, as previously described.

In accordance with a specific example, the ring pieces 120 may be divided by barriers (not shown), configured for isolating the ring pieces 120 from one another. Under such a design, one of the breach gates 140 may be configured for being breached at a first location of the GI and for performing an immunoassay for the detection of a first substance, while another of the breach gates 140 may be configured for being breached at a second location of the GI, different from the first location, for performing an immunoassay for the detection of a second substance, different from the first substance. Since the ring pieces 140 are isolated from one another, different immunoassay processes can be performed, independently, in different sections of the GI tract.

It is noted that the above described configuration provides, inter alia, the advantage of easy assembly, as each of the ring pieces 120 can be assembled individually, and it provides complete access to the assembler for inserting the LFS into the slots 125, 127. This is contrary to common in-vivo devices in which the LFS needs to be inserted or pushed-in into a narrow channel, when the in-vivo device is already half-assembled.

Turning now to FIGS. 4A to 4C, a breach film is shown generally designated 200, in the form of a thin film 202 having a thickness t and dimensions L×W (not marked). The film 202 comprises:
a cut-off active material having a threshold response to a specific substance of the GI fluid or to a specific parameter of the GI fluid;
a plasticizer configured, together with the cut-off sensitive material, for forming said film layer; and
an auxiliary active material.

The breach film 202 is configured for being exposed to GI fluids GF and for reacting with a certain substance or under certain conditions of the GI, only above a given threshold (either a given concentration of the substance or a level of a certain parameter, e.g. pH).

As shown in FIG. 4B, when the breach film 202 is under conditions which are below the threshold, the film 202 does not react with the GI fluids, and does not allow slow diffusion into the inlet 128. However, during exposure to the GI fluids GF, the film 202 may retain water therein, owing to the auxiliary active material.

Turning now to FIG. 4C, when the conditions of the GI fluids GF are above the predetermined threshold with which the film is configured to react, the film 202 is breached almost instantly (as it is already containing a great amount of water), and allows passage of the GI fluids GF into the inlet 128 and from there to the LFS. In this sense, the breach film 202 is configured for functioning as a cut-off breach gate 200, rather than allowing slow diffusion of fluids into the inlet 128.

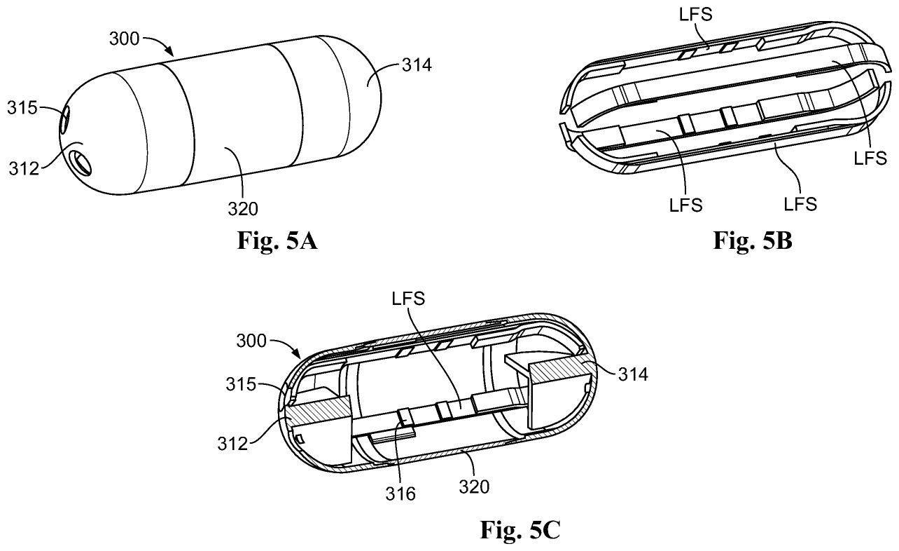

Attention is now drawn to FIGS. 5A to 5C, in which another example of an in-vivo device is shown, generally designated 300. The in-vivo device comprises a first end cap 312, a second end cap 314 and an intermediate shell piece 320 interposed between the two caps 312, 314. The first end cap 312 is formed with two inlets 315 configured for allowing ingress of GI fluids into the cavity of the in-vivo device to come into contact with the lateral flow strips LFS accommodated therein.

With particular reference being made to FIGS. 5B and 5C, the in-vivo device 300 comprises four lateral flow strip LFS, arranged symmetrically about the central axis of the in-vivo device 300. Each of the strips LFS extends the entire length of the in-vivo device 300, with its sample pad located proximal to the inlets 315. Such an arrangement positions the test bands 316 close to the center of the in-vivo device 300, at the widest section thereof, providing the maximal space for a sensor/imager (not shown) to be placed within the in-vivo device facing the bands 316.

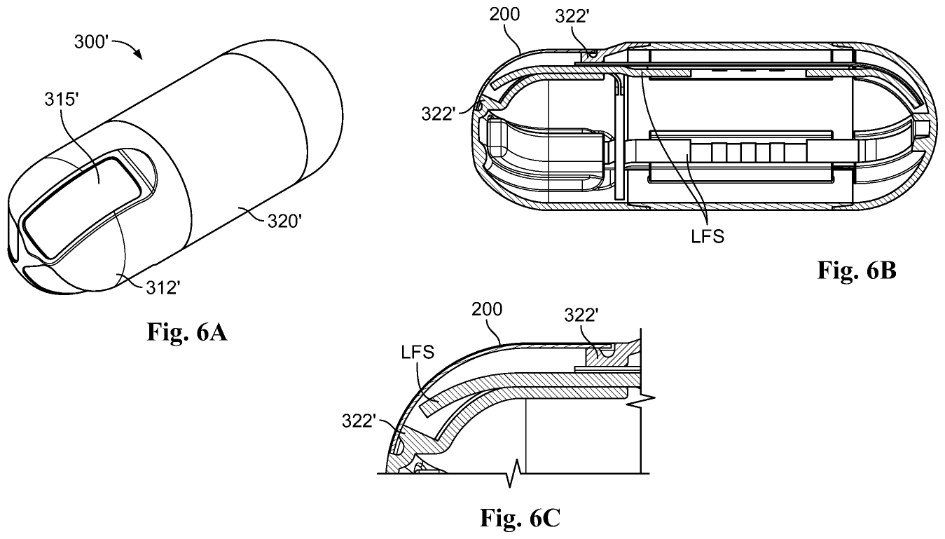

Turning now to FIGS. 6A to 6C, a variation on the in-vivo device 300 is shown, generally designated 300′. The in-vivo device 300′ differs from the device 300 in the geometry of the inlets 315, specifically, the inlets 315′ are designed to have a curvature only about one axis, compared to the inlets 315 which have a spherical surface. This may be particularly useful when using the breach film of the present invention, as it eliminates the need of the breach film, which is generally flat, to assume a spherical configuration. Instead, when using the inlets 315′, the breach film merely needs to bend in a single direction, allowing a more convenient fitting of the breach film to the shell.

In particular, the inlets 315′ are designed to be indented within the shell and having the desired geometry, such that they are not affected by the overall spherical geometry of the first end cap 312′. As such, the breach film 200 can be neatly placed onto the support 322 and have the edges thereof properly adhered to the supports 322 without any undesired crimps or creases.

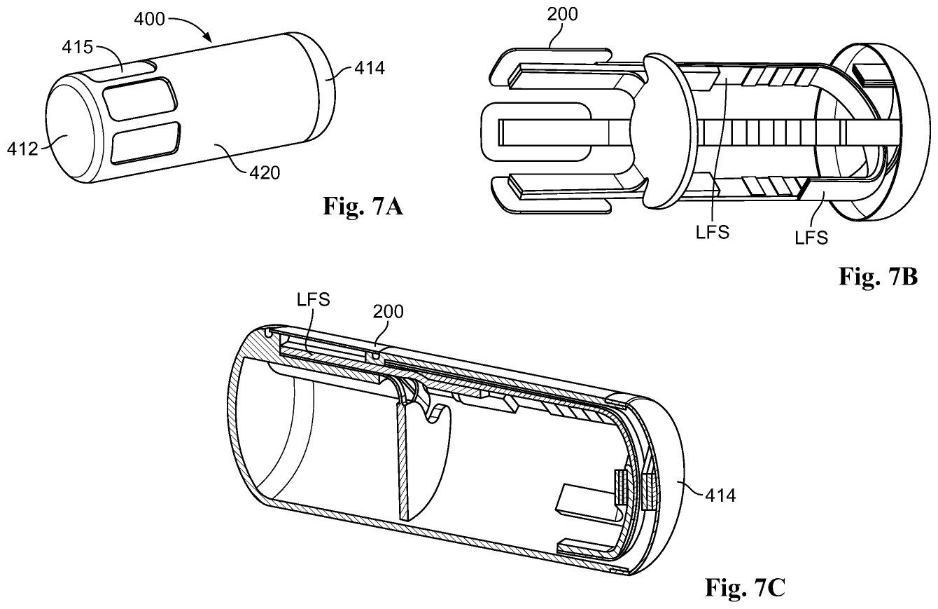

Attention is now drawn to FIGS. 7A to 7C, in which yet another example of an in-vivo device is shown, generally designated as 400 and comprising a shell 412 and an end cap 414, accommodating therein three lateral flow strips LFS, and three breach films 200, sealing off three corresponding inlets 415.

In the present example, the lateral flow strips LFS are arranged along the body of the in-vivo device 400, with one of their ends, containing the sample pad, juxtaposed with the inlet 415, and the other of their ends being curved across the end cap 414. This configuration may be particularly useful for longer lateral flow strips LFS which cannot fit in their entirety into the limited length of the in-vivo device 400.

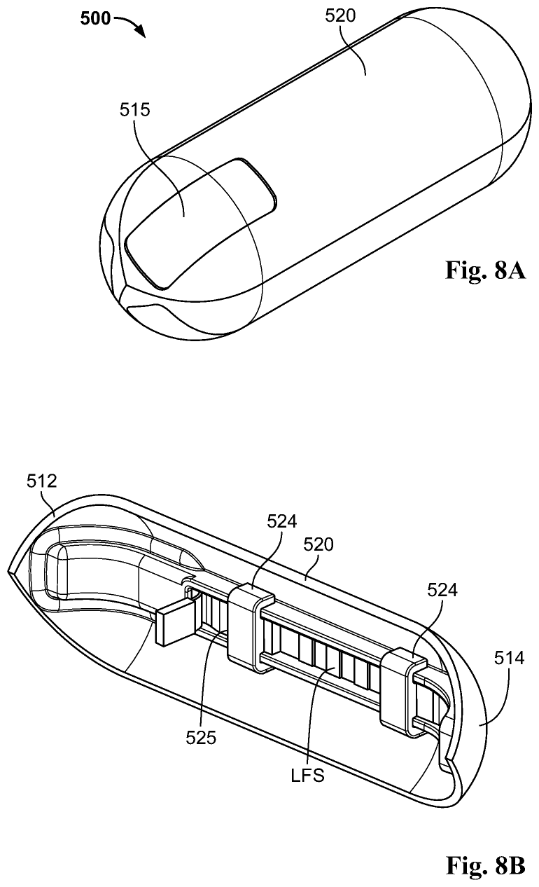

Finally, attention is drawn to FIGS. 8A and 8B, in which another configuration of an in-vivo device is shown, generally designated 500 and comprising a shell made of three shell pieces 520. Similarly to the previously described in-vivo device of FIGS. 3A to 3D, in the present example, the shell pieces 520 are longitudinal, each extending the entire length of the in-vivo device 500, and comprising a part of the first end dome 512 and a part of the second end dome 514. When assembled, the parts of the first and second end domes 512, 514 of the individual shell pieces 520 form together the first and second domes.

In addition, each shell piece 520 is formed with two brackets 524 spaced from an inner wall of the shell piece 520, forming a slot 525, sufficient for placing therein a lateral flow strip LFS. Thus, the current example provides similar advantages as those of the ring pieces 120 previously shown, allowing convenient access to an assembler of the in-vivo device 500.

The geometry and configuration of the inlets 515 is similar to that previously shown with respect to FIGS. 6A to 6C.

Those skilled in the art to which this invention pertains will readily appreciate that numerous changes, variations, and modifications can be made without departing from the scope of the invention, mutatis mutandis.

It will thus be seen that the objects set forth elsewhere herein, among those made apparent from the preceding description, are efficiently attained and, because certain changes may be made in carrying out the method described elsewhere herein and in the construction(s) set forth without departing from the spirit and scope of the invention, it is intended that all matter contained in the above description and shown in the accompanying drawings shall be interpreted as illustrative and not in a limiting sense.

In the foregoing detailed description, numerous specific details are set forth in order to provide an understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments.

Although embodiments of the invention are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

It is also to be understood that the following claims are intended to cover all of the generic and specific features of the invention herein described and all statements of the scope of the invention which, as a matter of language, might be said to fall therebetween.

1. A swallowable in-vivo device, comprising:
a shell defining a cavity of the in-vivo device, the shell being formed with at least one aperture extending therethrough and configured for allowing ingress of fluid into the cavity;
an immunoassay system disposed within the cavity and configured for interacting with fluid; and
at least one breach mechanism covering the at least one aperture for preventing ingress of fluid into the cavity via the inlet, the at least one breach mechanism including a film layer configured for reacting with the fluid and configured to be breached after a predetermined amount of exposure time to gastrointestinal (GI) fluid of a person, the predetermined amount of exposure time corresponding to travel of the in-vivo device along the person's GI tract to a desired location along the person's GI tract.

2-3. (canceled)

4. The swallowable in-vivo device according to claim 1, wherein the film layer is formed as a stand-alone component.

5. (canceled)

6. The swallowable in-vivo device according to claim 1, wherein the film layer is configured to erode over a period of exposure time to the GI fluid.

7. The swallowable in-vivo device according to claim 1, wherein the film layer is configured to prevent ingress of fluid into the inlet before the film layer is breached.

8. The swallowable in-vivo device according to claim 1, wherein the film layer comprises a combination of at least the following materials:
a cut-off active material having a threshold response to a specific substance of the GI fluid or to a specific parameter of the GI fluid;
a plasticizer configured, together with the cut-off active material, for forming the film layer; and
an auxiliary active material.

9. The swallowable in-vivo device according to claim 8, wherein the cut-off active material is an enteric material configured for reacting with the GI fluid.

10. The swallowable in-vivo device according to claim 8, wherein the cut-off active material remains inactive until a pH level of the GI fluid is one of under or over a threshold pH level.

11. The swallowable in-vivo device according to claim 1, wherein the film layer contains between 40-98% of enteric material.

12. The swallowable in-vivo device according to claim 8, wherein the plasticizer is selected from the group consisting of triethyl citrate glycols, diesters and triesters of acids, diesters and triesters of alcohols, natural oils, and Polyethylene Glycols.

13. The swallowable in-vivo device according to claim 12, wherein the plasticizer is propylene glycol or polyethylene glycol (PEG).

14. The swallowable in-vivo device according to claim 9, wherein the amount of plasticizer in the film layer is the complement of the enteric material to 100%.

15. The swallowable in-vivo device according to claim 8, wherein the amount of plasticizer in the film layer ranges from 60-2%.

16. The swallowable in-vivo device according to claim 8, wherein the auxiliary active material is configured for providing the film layer with additional resilience.

17. The swallowable in-vivo device according to claim 8, wherein the auxiliary active material is a hydro-swelling enteric polymer configured for retaining fluid within the film layer before the film layer is breached.

18. The swallowable in-vivo device according to claim 1, wherein the film layer functions as an indicator that the in-vivo device has reached a desired location along the GI tract.

19. The swallowable in-vivo device according to claim 18, wherein the in-vivo device comprises a sensor behind the film layer, and the film layer is configured to be breached at the desired location along the GI tract, wherein, when the film layer is breached, the fluid triggers the sensor, thereby indicating that the in-vivo device has reached a desired location.

20. The swallowable in-vivo device according to claim 16, wherein the amount of auxiliary active material is provided as being between 2-40% of the combined weight of the cut-off material and the plasticizer.

21-24. (canceled)

25. A film layer configured for being used in an in-vivo device, the film layer comprising:
a cut-off active material having a threshold reaction to a substance or parameter of gastrointestinal (GI) fluid of a person;
a plasticizer configured, together with the cut-off sensitive material, for forming the film layer; and
an auxiliary active material, wherein the film layer is configured to be breached after a predetermined amount of time of exposure to the GI fluid.

26-31. (canceled)

32. A swallowable in-vivo immunoassay device, the immunoassay device comprising:
a lateral flow strip extending between a fluid intake end and a distal end, the lateral flow strip including a test pad and a backing card juxtaposed with the test pad, wherein the backing card is made of a material allowing at least partial passage of light therethrough;
an illumination module including at least one illumination source configured for directing light towards the backing card; and
a sensor module including at least one sensor configured for receiving light from the illumination module, wherein the at least one sensor is positioned such that the test pad is disposed between the at least one sensor and the backing card.

33. The swallowable in-vivo immunoassay device according to claim 32, wherein the test pad includes a test band configured to react with gastrointestinal fluid of a person, thereby changing at least one property of the test band.

34-58. (canceled)
